(19) United States
(12) Patent Application Publication
(10) Pub. No.: US 2023/0054890 A1
(43) Pub. Date: Feb. 23, 2023

(54) Automated Musculoskeletal Physical Therapy Collaborative Systemic Delta System

(71) Applicant: ELLIOT RAJ, TAMPA, FL (US)

(72) Inventor: ELLIOT RAJ, TAMPA, FL (US)

(21) Appl. No.: 17/405,970

(22) Filed: Aug. 18, 2021

(51) Int. Cl. G16H 20/30; G16H 40/60; G16H 10/60; G16H 15/00

(52) U.S. Cl. CPC G16H 20/30; G16H 40/60; G16H 10/60; G16H 15/00

(57) ABSTRACT

A system and method for administering musculoskeletal physical therapy automatically to patients is disclosed. The apparatus is comprised of a systemic device having articulating arms with interchangeable heads, an interactive touch screen and cloud network. Physical therapists input treatment plans and calibrate the device to each patient and activate the treatment process under careful supervision. Said devices come in a range of sizes, multiple arms and operate on multiple axis depending on the scope and nature of treatment. Sensors in the heads determine treatment progress and collect data. Once a treatment is completed, this data is furnished to third parties such as doctors and insurance providers. An object of the invention is to automate the process of lengthening fascia tissues and minimizing adhesion in muscular injuries as well as to track patient progress and automate related reporting requirements.

### FIELD OF THE INVENTION

The invention relates to physical therapy techniques. More specifically, it relates to a system and method for administering musculoskeletal physical therapy automatically to patients.

### BACKGROUND

Fascia is a tough, continuous, connective tissue throughout the body in a three-dimensional web from head to toe without interruption. Trauma, posture, or inflammation can create a binding of the fascia resulting in excessive pressure on nerves, muscles, blood vessels, osseous structures and internal organs. While fascia is the “casing” that holds out muscles in a protective continuous membrane with a nervous system that is not completely understood by science today, it is the actual muscular system inside this casing that presents the challenges to proper musculoskeletal health, and more importantly, the best opportunity to fix musculoskeletal abnormalities, whether they come from age and overuse, or genetics and/or disease. We are absolutely convinced that severing or cutting of any portion of the continuous fascia system is damaging to the human systems beyond what medicine can currently measure, we know if it is not absolutely necessary it should not be done. Since many standard tests such as CAT Scans, X-Rays, myelograms, electromyography, etc. do fail to completely represent fascia and musculoskeletal restrictions it is thought that an extremely high percentage of people suffering with pain and a lack of motion may be having fascia and musculoskeletal problems and most go undiagnosed and treatments applied are usually not the most effective. An example of this is a chiropractor who chooses to continuously “adjust” the spine, without proper muscular analysis can never fix the true problem, and in fact is applying pressure and additional inflammation to joints that cannot be adjusted without addressing the true underlying cause of the misalignment they seek to fix. Unless the muscle is addressed it will NEVER allow the bones to be properly aligned. Many times supplements and elctro therapy are further applied, none of which can fix the real issues causing the symptoms. Chiropractics in recent years have begun to understand this and many are applying more musculoskeletal treatments in their practices. Another example is muscles that are so tight that are causing either joint or body alignment issues to the point where doctors prescribe surgical means to not only cut through the fascia, which we believe to be much more damaging then ever thought, but also to cut the muscle, and using human-made fibers, to lengthen the muscle. While these techniques have proved successful, we see them as great overkill. If one can use 100% “non-invasive” techniques to not only not disturb the fascia in any negative way (but in fact make it more healthy as we apply the process), but also press deep into every layer of the underlying muscles until true lengthening of the muscle occurs, then the patient does not have not only not have surgery, they do not have to suffer all the negatives of invasive techniques including long recovery periods, anesthesia, pain medications, on and on and on. Furthermore, Fascia, in its three dimensional and continuous web throughout the body, contains a network of nerves that are not yet completely understood. Many surgeries to correct medically-related diseases and disorders, pains, and afflictions cut through the fascia and make very invasive attempts to treat other ailments. Since we do not fully understand the purpose of this fascia connectivity, we do not understand the total effect it has on body systems when these are lacerated and in some cases removed large pieces. As technology and internet of things becomes more popular, the health car industry has started looking at ways to leverage systemics to compliment physical therapy treatments. United States Patent No. U.S. Pat. No. 5,466,213A granted to Neville Hogan, Hermano I. Krebs, Andre Sharon, and Jain Charnnarong disclosed a system that guides patient's limbs through a series of desired exercises with a systemic arm. United States Patent No. U.S. Pat. No. 5,466,213A granted to Neville Hogan, Hermano I. Krebs, Andre Sharon, and Jain Charnnarong also disclosed an interactive systemic therapist system that guides patients through rehabilitative exercises. Chen et. al. (2016) disclosed an assistive control system with a special kinematic structure of an upper limb rehabilitation system embedded with force/torque sensors. While many such systems have been established that allow for guided physical therapy, no systems were found that claim to provide automated physical therapy with autonomously-guided pressure-based treatments for lengthening fascia and muscular tissues and minimizing adhesion in muscular injuries and conditions.

### SUMMARY OF THE INVENTION

The device herein disclosed and described provides a solution to the shortcomings in the prior art through the disclosure of a system and method for administering musculoskeletal physical therapy automatically to patients. An object of the invention is to stream line the evaluation and patient intake process. During a treatment, a doctor refers a patient to a physical therapist who first has a patient complete Pain Self-Efficacy Questionnaires (such as the Short-Form McGill Pain Questionnaire, the Short-Form McGill Pain Questionnaire 2 and the Brief Pain Inventory-Short Form etc.). These forms can be administered using software on compatible evaluation tablets that also capture and record goniometer data visually (patient limb angles and ranges of motion). For example, an evaluation tablet records a patient's ability to raise and lower their head using tablet image processing. These images and angle data are immediately transmitted to the device wirelessly. Conversely, a therapist can take intake data and goniometer measurements and input them into the device manually by means of an onboard touch screen.

Another object of the invention is to make treatment recommendations specific to the patient's immediate needs. Once an evaluation is completed, the system uses Artificial Intelligence (AI) algorithms and recommends the appropriate treatment plan that includes assigning a unique patient identification (that allows for patient anonymity), probe head selections, pressure settings, temperature settings, electro-stimulus settings, and time durations etc. to the physical therapist. This AI has been programmed to be self-perpetuating as it continues to record patient data and treatments while absolutely protecting patient anonymity.

Another object of the invention is to provide specialized interchangeable probe heads that come in various shapes and sizes depending on the treatment area and nature of the fascia and muscles involved. For example, a large treatment on a patient's back might involve (large diameter probe heads) quick assembly of the smaller modular heads into a larger treatment head that matches the muscle areas to be treated, with a deeper surface relief than a small treatment area on a patient's foot that might involve a more pointed probe head with a shallow relief etc. To be more specific, the “depth” of the treatment is controlled by the pressure settings, and in one head attachment that is smaller than the standard 3 cm modular pieces and is pointed to get into highly directed areas such as the acromion process. These probe heads are interchangeable can be swapped out by the therapist. The therapist usually palpates a patient's muscles to do final determination of muscular sensitivity to pressure due to pain and adhesion and performs the human alignment into the device., and the system assigns range of motion scores, pain scales, range of motion at related joints based on the data collection processes performed by patient, therapist, and system just prior to treatment, and the system also adds patient historical data into the final treatment plan for that session.

Another object of the invention is to allow for autonomous treatment for various locations on a patient's body. The device is equipped with an (multitude of) articulating arm(s) that the probe heads are affixed. There are several models with different arms, with the only difference being the number of axis and the length of arm segments. The purpose of each model and the number of segments and lengths are detail further within this document, but it can be said that the models range from 2 axis up to 7 axis arms. These arms swivel at various joints, are electronically-powered and are available in many degrees of freedom with different axes. In addition, the device is mounted in various ways-for example a small device can be wall mounted and focus on limb treatments and a full-size device can be mounted on wheels. The device is available in a wall-mounted version for static, spot usage where patients either sit on a chair or lay on table for treatments. A cart-mounted device is available that allows for movement within a clinic, hospital, or other treatment centers, and also allows for movement between locations. Small wall mounted “specialty” devices can be used for procedures such as pre and post-surgical treatments in podiatry centers and hospitals. The smaller devices are not intended to treat the entire body-but rather one localized area. Smaller sized devices are for specialty disciplines of medicine that focus on specific areas of body that require musculoskeletal physical therapy.

Another object of the invention is to allow for different treatment probe head movement patterns and pressures to be performed. Once the device generate a treatment plan, the therapist can preview the activity on the software and see pressures represented as a heat map as well as the patterns generated by the probe before treatment begins on a patient. The Therapist has the ability to create waypoints to set or over-ride a treatment plan/area. This process is used when large muscles such as hamstring or pectoral muscle is selected for treatment and the therapist chooses to spot treat specific spots on the muscle that have inordinate amount of adhesion, and/or are much thicker than other parts of a large muscle, thus they require more treatment and deeper treatment than those other areas. Each waypoint can be programmed to use different pressures, temperature, electrical and time duration as the system moves though the treatment session for that muscle. Or it can be a simple path that is followed and repeated at the same pressures, temperature, electrical, and duration. Probe pressure can initially be set via a voice recognition option by therapist or manually input using the tablet or a touch screen on the device. The voice recognition allows the patient to set adjust starting point by saying “delta treatment lower” or higher as the case may be, and the device will adjust less pressure-if the device's pressure is too aggressive for a patients pain threshold. Depending on the recommended treatment prescribed by the device software, the device can perform rotational, vibrational, and pulsating pressures etc. to achieve fascia and muscle elongation. The device has a methodical instruction method that allow probe heads to pushing slowly, over and over deeper and deeper into each layer of the muscle, thereby truly lengthening the fascia and muscles. The probes are in contact with the skin for extended duration and each “touch” records a data set throughout the treatment session and pressure data, skin/muscle temperature data, and electromagnetic states continuously work together to lengthen and remove adhesion, along with returning proper alignment and flexibility, increased blood flow, and joint pressure reduction to an effected area.

Another object of the invention is to allow the device to measure treatment progress. As treatments are performed, sensors in the probe heads measure variables such as temperature, electromagnetic state, pressure, and probe accelerations etc. (other embodiments of the invention can utilize other sensors that will be discussed in more detail in the detailed discussion section of this disclosure). Over time, the device records data from these sensors and another evaluation is performed by the therapist using the compatible tablet system (as previously discussed). All of this data is analyzed by the system software that is connected wirelessly to a cloud network. AI algorithms in the network interpolate the findings and report treatment progresses back the physical therapist. Changes in data such as increased muscle temperature, higher pressure settings, and recording of changes in electrical states are all recorded, and those findings indicate a healthy reaction to a treatment (via increased blood flow) and reduced muscle temperature findings would signify an issue has yet to be resolve and reevaluation may be necessary. Treatment can also be considered a success when measured in conjunction with the other compatible range of motion software with tablet that uses camera and machine and deep learning AI models to not only give front and side alignment assessments but also generates anomaly reports (as previously mentioned).

Another object of the invention is to allow a therapist to configure the device to dynamically change treatments on the fly autonomously. For example, the device detects that a set of fascia and muscle is being treated successfully and is exhibiting the signs of improvement. The device can switch probe heads automatically and continue a more invasive treatment to promote continuous healing on a patient in real time. The application of increased pressure or increased duration signifies increased muscle health, and can be validated by manual palpation, patient pain level self-assessment and post treatment range of motion testing performed by the system.

Another object of the invention is to allow for redundancy in safety measures. The device contains at least three kill switches at different locations that are easily within reach of the patient and the therapist. Other safety measures include bump sensors, pressure and acceleration sensors that automatically halt treatments immediately when preset thresholds are detected.

Another object of the invention is to provide automatic record keeping for treatments performed. The device software is compatible with many popular physical therapy programs. In addition, the cloud network contains proprietary software that gathers detailed treatment activities that are collect from the device in real time and tracks patient progress for third parties such as a patient's primary health care provider and their insurer, but it also copies patient data removing patient specific personal information but keeping all demographics and treatment data. The data is used to power the AI deep learning modules of the Delta System Systems.

Another object of the invention is to provide direct and precise muscle stimulation. Electrical stimulation from the device includes voltage levels between 0 and 100 volts which is more powerful than the widely known Tens Units but less powerful than requiring a prescription from a Doctor and does not require FDA approval. This voltage is utilized for two reasons: first the potential side effects or damage to nearby systems is limited; second, this level of voltage can get very deep into the muscle (and on top of the subject area) requiring less electrical current to be effective. Subsequently, the device is considered to be much safer and more of a “directed” and “precise” therapy.

Another object of the invention is to provide controlled vibrations to muscles. The deep penetrating, non-invasive linear pressure can be supplemented with very mild vibrations and very minimal rotational pressures by means of the various nodes. Vibrational therapy is only capable of penetrating scant millimeters from the skins surface. Once the current device penetrates deeply into a muscle or past organs or other systems, the device applies minimal vibrational or rotational pressures very close to the area requiring deep treatment. This device limits the amount of vibrational and rotational pressures that are generally used in automatic systems (which often have several types of adverse effects to derma, and adjacent systems).

It is briefly noted that upon a reading this disclosure, those skilled in the art will recognize various means for carrying out these intended features of the invention. As such it is to be understood that other methods, applications and systems adapted to the task may be configured to carry out these features and are therefore considered to be within the scope and intent of the present invention, and are anticipated. With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention. As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The objects features, and advantages of the present invention, as well as the advantages thereof over existing prior art, which will become apparent from the description to follow, are accomplished by the improvements described in this specification and hereinafter described in the following detailed description which fully discloses the invention, but should not be considered as placing limitations thereon.

### BRIEF DESCRIPTION OF THE FIGURES

FIG. 1 showing a perspective view of the device.

FIG. 2 showing the onboard tablet with interactive touch screen.

FIG. 3 showing another embodiment of the device.

FIG. 4 showing a representative view of the device's interconnections.

FIG. 5 showing a representative view of the device's method.

FIG. 6 showing a perspective view of another embodiment of the interchangeable head.

FIG. 7 showing a perspective view of another embodiment of the interchangeable head.

FIG. 8 showing a perspective view of another embodiment of the interchangeable head.

FIG. 9 showing a perspective view of another embodiment of the interchangeable head.

FIG. 10 showing a perspective view of another embodiment of the interchangeable head.

### DETAILED DESCRIPTION OF THE FIGURES

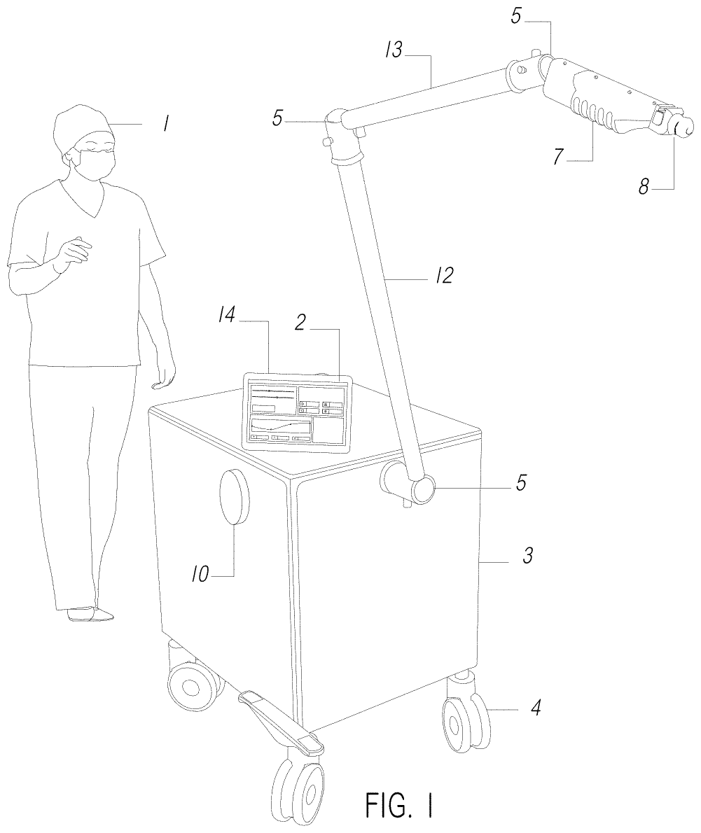

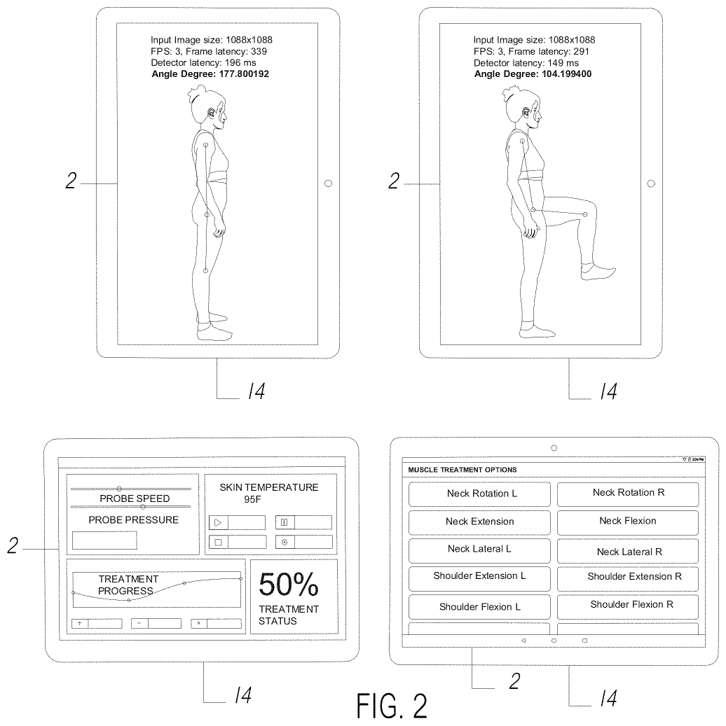

FIG. 1 showing a preferred embodiment of the device comprised of square-shaped cart 3 being operated by physical therapist 1 and being made of a rigid material such as metal and the like and containing components including but not limited to: electric motors; rechargeable batteries (lithium ion, nickel hydride etc.); a micro controller with onboard memory and wireless WiFi transmitter and connected to an onboard tablet 14 removably-secured to cart 3. Said onboard tablet 14 has a touch screen utilizing an operating system such as Android, Apple etc. with wireless system communicating with a statically-mounted Linux board that is housed in the micro controller housing. The device also having voice recognition capabilities allowing both physical therapists and patients to command pre-assigned operations. The onboard tablet 14 also having functions such as emergency kill switch 10 and other control buttons such as but not limited to on/off etc. Other embodiments of cart 3 may have hydraulic motors, hydraulic reservoirs and compressed air pump and air tanks. Onboard tablet 14 having software application on a non-transitory computer readable medium including computer readable instructions (to be discussed in FIG. 2).

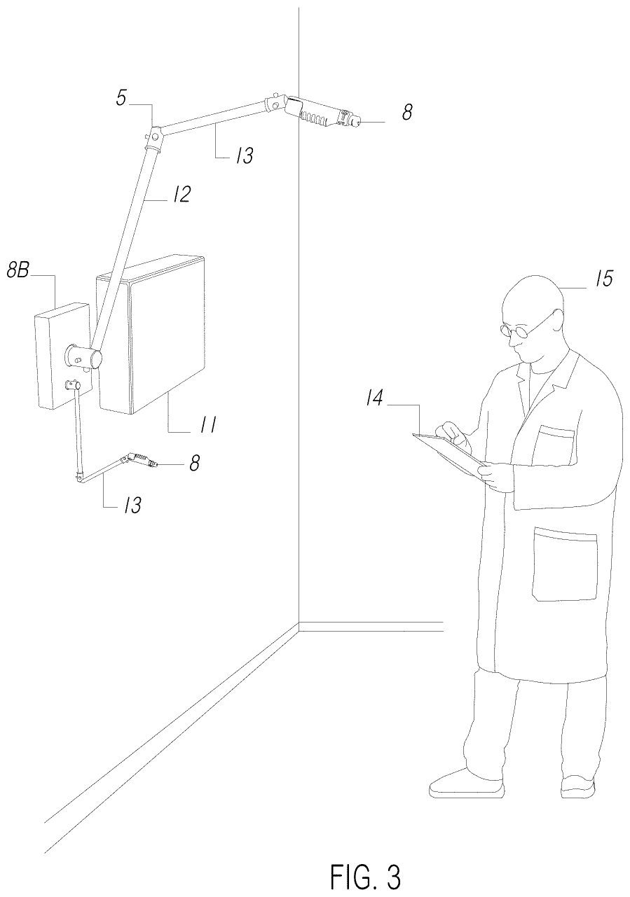

Said cart 3 having a plurality of tubular-shaped, upper articulating arms 13 and lower articulating arms 12 pivotably-connected to the cart on one distal end and connected to probes 7 on another distal end. The aforementioned arms having internal mechanisms that allow them to articulate that include but are not limited to stepper motors, linear actuators, hydraulic rams, pneumatic cylinders etc. configured inside swivel joints. Said software within microcontroller governing speeds of stepper motors and linear actuators etc. to at least 250 mm per second or less. The arms 12 and 13 being connected to one another by means of said swivel joints 5 that allow said arms to rotate and articulate in multiple axes. Said swivel joints 5 are either movable in a circular direction and others are moved in a Cartesian coordinate pattern. Other embodiments of the device have different numbers of axes depending on whether or not a treatment will be applied to a patient's whole body, or specific applications-such as podiatry focusing on just feet which would require only a small 3 axis device. The device having a number of articulating arms 12 and 13 that include but are not limited to between two to seven axes depending on the application as stated. Each distal end of upper articulating arms 13 being rigidly affixed to interchangeable heads 8 by means of the aforementioned probes 7. Probes 7 having internal, electrical motors and weighted vibration axles therein that can be programmed for various types of movements by the device software that include but are not limited to side-to-side motions, circular movements, pulsations and elliptical motions etc. FIG. 3 showing another wall mount embodiment of the device having a multitude of articulating arms as wells a s wall mount 8B and control cabinet 11 in communication with user 15 having the onboard tablet 14.

Said interchangeable heads 8 being comprised of cup-shaped flexible, swappable nodes of various sizes and having a plurality of reliefs including but not limited to ridges, bumps etc. The interchangeable heads 8 also having a multitude of embedded sensors including but not limited to: temperature; pressure; and motion detectors and the like. Other embodiments of the device can include interchangeable heads 8 such as pressure sensors, accelerometers, LIDAR, temperature readers and speed sensors. Said sensors send data to the device's microcontroller software which can then be transmitted wirelessly to cloud network 23 and interpreted into treatment evaluations and assessments that can be available for viewing on onboard tablet 14.

FIG. 2 showing various views of the onboard tablet 14 with software application 2 therein which uses the tablet's existing camera to capture digital images of a patient. Digital images then being analyzed by an algorithm that identifies joints on a patient's body automatically and superimposes pivot points based on a master list on said images and interpolates angles of range of motion as selected by said physical therapist. The user sending range of motion angle information to stakeholders that include but are not limited to other physical therapists, insurance companies and medical health care professionals and the like (doctors etc.). The figure also showing screens that include but are not limited to treatment status (probe activity, sensor readings, overall treatment status, and treatment time remaining etc.) and treatment selections (for neck, shoulders etc.) and the like. The invention app software 8 being written from code that may include, but not be limited to: Java, C++™, Visual Basic™, Fortran™, Basic™ and the like. The software also compatible with a plurality of operating systems such as, but not limited to: Windows™, Apple™, and Android™, and compatible with a multitude of hardware platforms such as, but not limited to: personal desktops 7, laptops, tablets, smartphones and the like. In the following description, for purposes of explanation, numerous specific details are set forth in order to provide a thorough understanding of the present invention. However, it will be apparent that the invention may be practiced without these specific details. In other instances, well-known structures and devices are shown in block diagram form in order to facilitate describing the present invention. As used in this application, the terms “component”, “handler”, “model”, “system” and the like are used to refer to computer-related entities, hardware, a combination of hardware and software, software, or software at run time. For example, a component may be, but is not limited to being, a process running on a processor, a processor, an object, an executable, a thread of execution, a program, and a computer.

By way of illustration, both an application running on a server and a server can be a component One or more components can reside within a processor and thread of execution and a component can be localized on one computer and distributed between two or more components. In addition, these components can execute from various computer readable media having various data structures stored thereon. Components may, for example, interact with other systems across a network such as the Internet via one or more data packets (data from one component, and signals that interact with components of other systems in a local or distributed system). Communication via local and remote processes in accordance with signals with data from components). The computer component may be stored, for example, on a computer readable medium, which may be an application specific integrated circuit (ASIC), compact disc (CD), digital video disk (DVD), ROM (ROM) according to the present invention. Read Only Memory, floppy disk, hard disk, electrically erasable programmable read only memory (EEPROM), and memory sticks.

In view of the disclosure provided herein, a mobile application is created by techniques known to those of skill in the art using hardware, languages, and development environments known to the art Those of skill in the art will recognize that mobile applications are written in several languages include, by way of non-limiting examples, C, C++, C#, Objective-C, Java, Javascript, Pascal, Object Pascal, Python, Ruby, VB.NET, WTML, and XHTML/HTML with or without CSS, or combinations thereof. The software also compatible with a plurality of operating systems such as, but not limited to: Windows, Apple, and Android, and are compatible with a multitude of hardware platforms such as, but not limited to: personal desktops, laptops, tablets, smartphones and the like. Suitable mobile application development environments are available from several sources. Commercially available development environments include, by way of non-limiting examples, AirplaySDK, alcheMo, Appcelerator, Celsius, Bedrock, Flash Lite, .NET, Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android, SDK, BlackBerry,SDK, BREW SDK, Palm OS SDK, Symbian SDK, webOS SDK, and Windows Mobile SDK.. Those of skill in the art will recognize that several commercial forums are available for distribution of mobile applications including, by way of non-limiting examples, Apple, App Store, Google Play, Chrome Web Store, BlackBerry App World, App Store for Palm devices, App Catalog for webOS, Windows Marketplace for Mobile, Ovi Store for Nokia devices, Samsung Apps, and Nintendo DSi Shop etc.

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java, Lisp, Python, Visual Basic, and VB .NET, or combinations thereof. Compilation is often performed, at least in part, to create an executable program. In some embodiments, a computer program includes one or more executable complied applications.

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe Flash Player, Microsoft Silverlight, and Apple QuickTime.

In view of the disclosure provided herein, those of skill in the art will recognize that several plug-in frameworks are available that enable development of plug-ins in various programming languages, including, by way of non-limiting examples, C++, Delphi, Java, PHP, Python, and VB .NET, or combinations thereof. Web browsers (also called Internet browsers) are software applications, designed for use with network-connected digital processing devices, for retrieving, presenting, and traversing information resources on the World Wide Web. Suitable web browsers include, by way of non-limiting examples, Microsoft Internet Explorer, Mozilla Firefox, Google Chrome, Apple Safari, Opera Software Opera, and KDE Konqueror. In some embodiments, the web browser is a mobile web browser. Mobile web browsers (also called micro-browsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google Android browser, RIM BlackBerry Browser, Apple Safari, Palm Blazer, Palm WebOS Browser, Mozilla Firefox for mobile, Microsoft Internet Explorer Mobile, Amazon Kindle Basic Web, Nokia Browser, Opera Software Opera Mobile, and Sony PSP browser. Software Modules

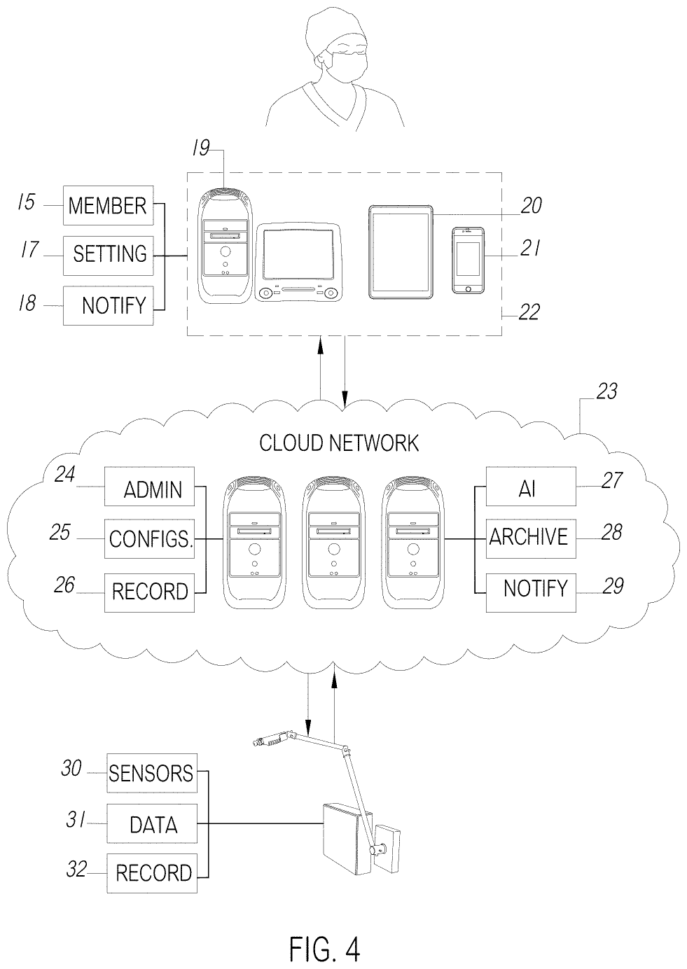

FIG. 4 showing a representative view of the device's interconnections wherein a user having the ability to perform operations including but not limited to membership entry 15 (login, licensing and practice demographics, patient information and records etc.); device settings 17 (pressures, durations, probe actions and patterns, temperature settings, sensor thresholds and ranges, treatment plans etc.); and notifications 18 (patient, doctor and insurance party notifications and communications) using computing devices 22 such as but not limited to desktop computers 19, conventional tablets 20 and mobile devices 21 etc. The device having operations that include but are not limited to sensor calibrations and emergency shutdown procedures 30; data collection and transmission (treatment and patient status etc.); data collection and transmission (patient fascia and muscle conditions such as temperature, treatment pressures and probe patterns etc.); and records 32 (treatment history and progression status etc.). The users and devices being connected by cloud network 23 having routines that include but are not limited to: administrative functions 24 (therapist activities and notes, patient records etc.); device configurations set by physical therapist 25 (probe pressures and patterns, sensor data types, treatment durations etc.); patient recordings 26 (intake images, treatment status etc.); artificial intelligence algorithms 27 (treatment outcome predictions etc.); archives 28 (encrypted data set libraries of patient data and treatment histories etc.); and notifications 29 (patient information transfer to health care providers and insurance companies via emails, text messages, SMS etc.). The system also uses odometry for location awareness, fault tolerance and logging.

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

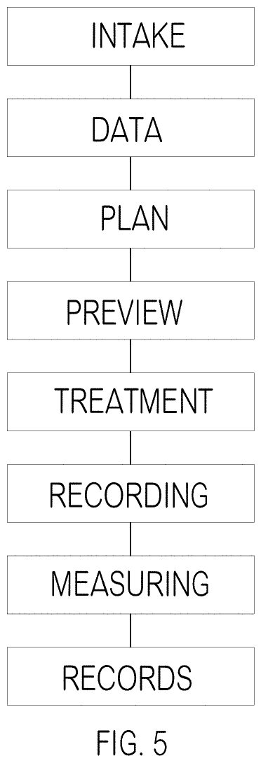

FIG. 5 showing the method of administering musculoskeletal physical therapy automatically to patients including but not limited to: completing patient intake using onboard tablet onboard tablet with interactive touch screen 14 (patient demographics, pain questionnaires, goniometer measurements, range of motion scores, pain scales etc.); developing treatment plan (probe selecting, setting pressure values, setting temperature values, establishing treatment durations, and setting waypoints, setting via voice recognition or manually with software); previewing treatment (heat map probe patterns, probe pressures etc.); performing treatment (autonomously applying pressure to elongate fascia and muscles in real time with linear, rotational, vibrational, and pulsating pressures etc.); recording physical treatment in real time (pressure, skin temp, electromagnetic level recordings); measuring progress (connecting data to cloud network, sending and receiving data, automatically and adjusting treatments based on data in real time dynamically) and keeping records (historical treatment activities, progression tracking, transmitting to health care provider and insurance company etc.).

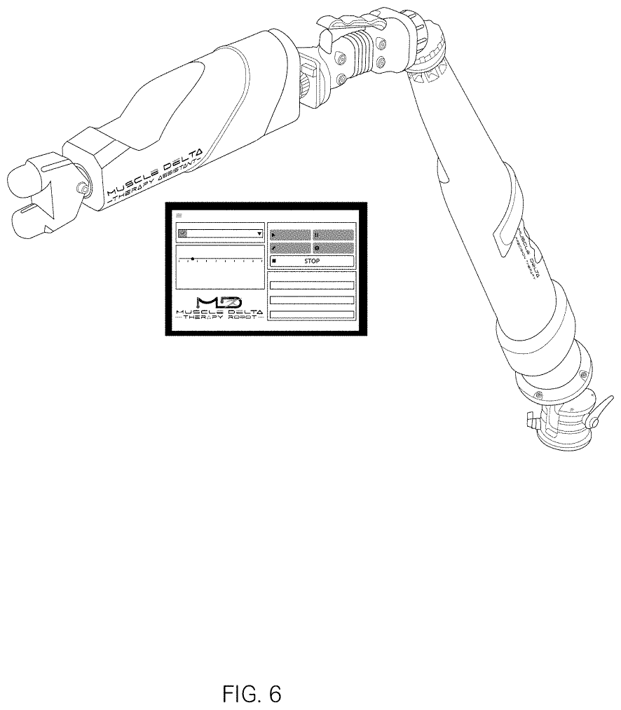

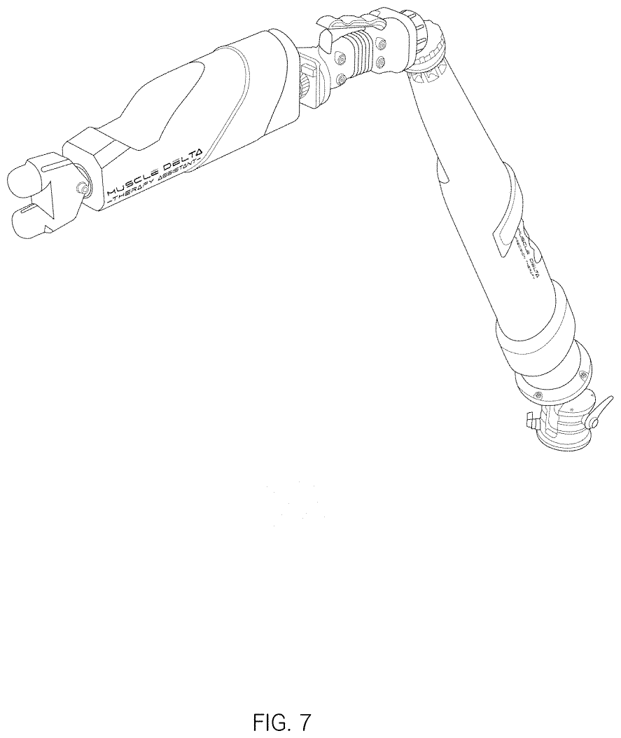

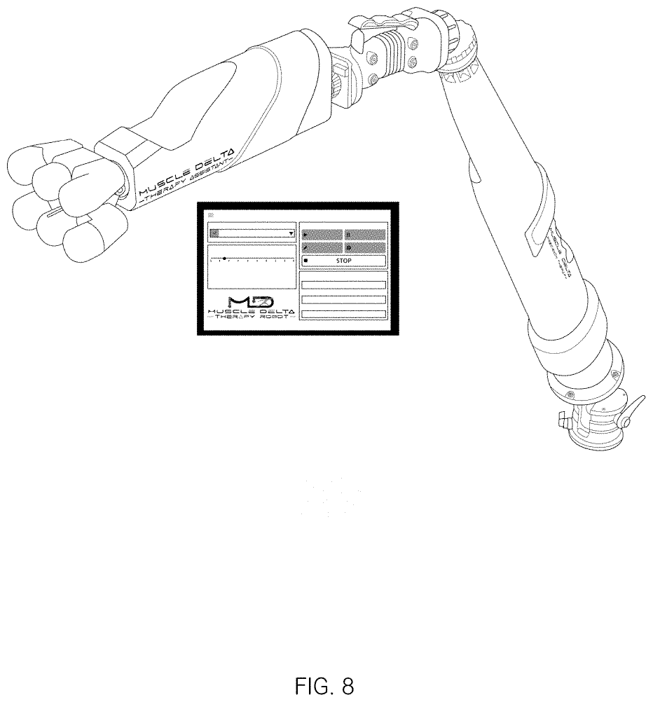

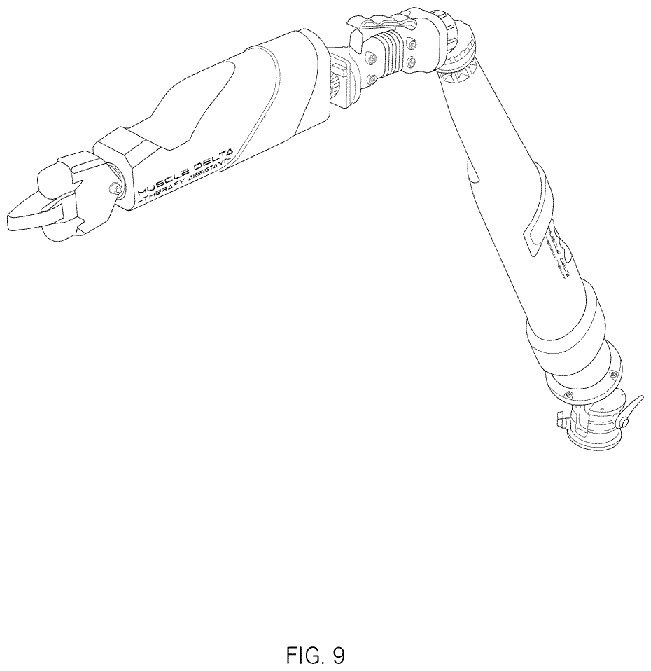

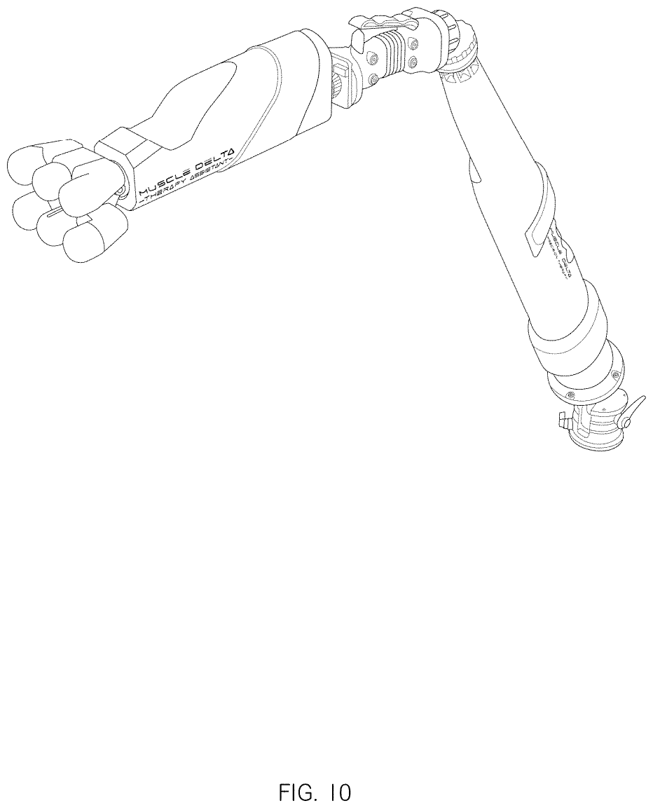

FIG. 6 showing a perspective view of another embodiment of the interchangeable head 8 with two nodes and tablet interface. FIG. 7 showing a perspective view of another embodiment of the interchangeable head 8 having two nodes. FIG. 8 showing a perspective view of another embodiment of the interchangeable head having six nodes and tablet interface. FIG. 9 showing a perspective view of another embodiment of the interchangeable head 8 with two nodes and center sensor. FIG. 10 showing a perspective view of another embodiment of the interchangeable head 8 having six nodes.

It is additionally noted and anticipated that although the device is shown in its most simple form, various components and aspects of the device may be differently shaped or slightly modified when forming the invention herein. As such those skilled in the art will appreciate the descriptions and depictions set forth in this disclosure or merely meant to portray examples of preferred modes within the overall scope and intent of the invention, and are not to be considered limiting in any manner. While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth.

What is claimed is:

1. A device for administering musculoskeletal physical therapy automatically to patients comprising:
a) a cart;
b) articulating arms pivotably-secured to the cart;
c) onboard tablet with interactive touch screen removably-secured to the cart;
e) probes affixed to the articulating arms;
f) interchangeable heads affixed to the probes;
g) a non-transitory computer readable medium including computer readable instructions;
h) cloud network connected to the device; and
i) onboard safety measures on the cart.

2. The device for administering musculoskeletal physical therapy automatically to patients of claim 1, wherein the cart further comprising wheels, rechargeable battery, and micro controller with memory and wireless transmitter.

3. The device for administering musculoskeletal physical therapy automatically to patients of claim 1, wherein the articulating arms further comprising stepper motors, linear actuators, hydraulic rams, pneumatic cylinders configured inside swivel joints.

4. The device for administering musculoskeletal physical therapy automatically to patients of claim 1, wherein the articulating arms being connected to one another by means of swivel joints and having axes that range from two to seven.

5. The device for administering musculoskeletal physical therapy automatically to patients of claim 1, wherein the probes further comprising internal, electrical motors and weighted vibration axles.

6. The device for administering musculoskeletal physical therapy automatically to patients of claim 1, wherein the interchangeable heads being comprised of various sizes, having a plurality of reliefs, and having embedded temperature, pressure, electrical, and motion detectors sensors.

7. The device for administering musculoskeletal physical therapy automatically to patients of claim 1, wherein the onboard safety measures further comprising bump, pressure, acceleration sensors, and LIDAR.

8. The device for administering musculoskeletal physical therapy automatically to patients of claim 1, wherein the non-transitory computer readable medium including computer readable instructions contains artificial intelligence algorithms that record patient data and treatments with anonymity, develops treatment plans for sessions, allows users to set treatment waypoints, uses odometry for location awareness, fault tolerance and logging and generate reports.

9. A method for administering musculoskeletal physical therapy automatically to patients comprising:
a) completing patient intake forms;
b) developing treatment plan;
c) previewing treatment plan;
d) performing treatment;
e) recording treatment in real time;
f) measuring treatment progress,
g) creating records and reports; and
h) keeping records.

10. The method for administering musculoskeletal physical therapy automatically of claim 9, wherein the completing patient intake forms includes the steps of obtaining patient demographics, completing pain questionnaires and recording range of motion scores.

11. The method for administering musculoskeletal physical therapy automatically of claim 9, wherein the developing treatment plan includes the steps of selecting probes, setting pressure values, setting temperature values, possibly setting waypoints, and establishing treatment durations using the onboard tablet with interactive touch screen.

12. The method for administering musculoskeletal physical therapy automatically of claim 9, wherein the previewing includes the step of viewing heat maps of probe patterns and pressures using the onboard tablet with interactive touch screen.

13. The method for administering musculoskeletal physical therapy automatically of claim 9, wherein the performing treatment includes the steps of the device probes autonomously applying pressure to elongate fascia and muscles with linear, rotational, vibrational, and pulsating pressures.

14. The method for administering musculoskeletal physical therapy automatically of claim 9, wherein the recording treatment in real time includes the step of recording probe pressure, skin and muscle temperature and electromagnetic levels.

15. The method for administering musculoskeletal physical therapy automatically of claim 9, wherein the measuring progress includes the step of connecting data to the cloud network, sending and receiving data automatically and adjusting treatments in real time.

16. The method for administering musculoskeletal physical therapy automatically of claim 8, wherein the keeping records includes the step of recording treatment activities, progression tracking, and transmitting records to health care providers and insurance companies.
